(19) United States
(12) Patent Application Publication
(10) Pub. No.: US 2009/0202666 A1
(43) Pub. Date: Aug. 13, 2009

(54) COSMETIC USE OF A CASSUMUNARIN, AN ARYLBUTENOID, AND/OR A BOTANICAL EXTRACT CONTAINING THEM

(75) Inventors: Serge Holderith, Biot (FR); Christelle Marie Suzanne Lasserre, New Jersey, NJ (US); Yannick Gérard Maestro, Martigues (FR)

Correspondence Address: YOUNG & THOMPSON, 209 Madison Street, Suite 500, ALEXANDRIA, VA, 22314, US

(73) Assignee: Chanel Parfums Beaute, Neuily Sur Seine Cedex (FR)

(21) Appl. No.: 12/304,660

(22) PCT Filed: May 31, 2007
(86) PCT No.: PCT/EP2007/055294

§ 371 Date: Dec. 12, 2008

Related U.S. Application Data
(60) Provisional application No. 60/831,668, filed on Jul. 19, 2006.

(30) Foreign Application Priority Data
Jun. 12, 2006 (EP) 06290953.6

(51) Int. Cl. A61K 36/906

(52) U.S. Cl. 424/756

(57) ABSTRACT

The present invention relates to a cosmetic skin care method to prevent and/or treat at least one sign of skin ageing, comprising the topical application to the skin of a composition comprising at least one active ingredient selected from cassumunarin and/or an arylbutenoid and/or a botanical extract containing them, such as an extract from Zingiber cassumunar Roxb. It also pertains to an extract from Zingiber cassumunar Roxb, which is obtainable by extracting rhizomes thereof with at least one apolar solvent having a polarity index of less than 1, optionally mixed with at least one polar solvent having a polarity index of more than 3.5.

The present invention relates to a cosmetic skin care method to combat at least one sign of skin ageing, comprising topical application to the skin of a composition comprising at least one cassumunarin and/or an arylbutenoid and/or a botanical extract containing them.

The skin is essentially constituted by three layers, namely the epidermis, the dermis, and the hypodermis, starting from the most superficial layer.

The epidermis is principally constituted by keratinocytes, and also by melanocytes (involved in skin pigmentation) and Langerhans cells, connected by intercellular lipids. Its function is to protect the body from the external environment and to ensure its integrity, and in particular to inhibit the penetration of micro-organisms or chemical substances and to prevent the evaporation of water contained in the skin, which would result in dehydration.

The dermis provides a solid support for the epidermis and also ensures its nutrition. It is essentially constituted by fibroblasts and an extracellular matrix principally constituted by collagen, elastin, and proteoglycans.

Elastin fibers are largely responsible for the elasticity and firmness of skin. They are initially synthesized by fibroblasts in the form of tropoelastin carrying lysine residues which then form elastin microfibrils by intramolecular bonding.

Elastin fibers tend to degrade with age, primarily due to the greater activity of elastases, resulting in slackening of the skin. Elastic fibers (elastose) are also observed to degrade under UV.

The search continues for novel active ingredients for cosmetics which can prevent or combat the signs of skin ageing, and in particular combat the loss of firmness of the skin.

In this regard, it is known that certain active ingredients such as ascorbic acid and its derivatives (French patent FR-A-2 847 816), ginsenosides and extracts of Panax containing them (FR-A-2 767 057), or extracts of Polygonatum (European patent EP-A-1 003 538), for example, can increase the synthesis of elastin by fibroblasts and can thus combat the loss of skin firmness.

However, there is a constant need for the provision of novel active ingredients for cosmetics which can more effectively combat the signs of skin ageing and in particular combat the loss of skin firmness.

Further, having regard to consumers' increasing interest in natural products including as few synthetic ingredients as possible, and to ever tighter regulations regarding industrially produced chemical compounds, it would be desirable for such active ingredients for cosmetics to be of plant origin.

The Applicant has now shown that certain active ingredients of plant origin, in particular phenylbutenoids and cassumunarins, have the property of increasing the synthesis of elastin by dermal fibroblasts.

Phenylbutenoids, extracted in particular from rhizomes of bengle (Zingiber cassumunar), are already known as cyclooxygenase-2 inhibitors [Han Ah-reum et al, Chemical & Pharmaceutical Bulletin (2005) 53(11), 1466-1468], anti-inflammatories and analgesics [Ozaki Yukihiro et al, Chemical & Pharmaceutical Bulletin (1991), 39(9), 2353-6], anti-oxidants [Masu et al, Phytochemistry, Vol. 39, No. 2, pp. 459-461, 1995] and anti-fungals [Picker et al, Journal of Ethnopharmacology 85 1003) 289-293].

The anti-oxidant and anti-inflammatory effect of cassumunarins extracted from Zingiber cassumunar has also been studied [Masuda et al, JAOCS, Vol. 72, No. 9 (1995)].

However, to the Applicant's knowledge, the use of arylbutenoids, cassumunarins, or botanical extracts containing them, especially certain bengle extracts, has never before been suggested for improving the firmness of the skin by topical application.

Bengle extracts are conventionally used in traditional Indonesian medicine (Jamu) to firm the skin. However, there is no indication or teaching that the extracts contain arylbutenoids and/or cassumunarins, nor that they are applied to the skin.

The use of aqueous or alcoholic extracts of that plant (preferably rhizomes) has also been suggested to combat the signs of skin ageing by inhibiting the activity of collagenases and elastases and by increasing the production of collagen (JP2003-176230) or as whitening or anti-pigmenting agents (JP09-16927 and JP09-071522). However, said extracts are not obtained using apolar organic solvents. In contrast, the extract described in Japanese application JP2003-176230 may be obtained after washing the rhizomes with hexane, the hexane extract then being eliminated.

Similarly, it has been suggested, in JP2001-192339, that bengle extracts be used as PAF antagonists to combat the desquamation and roughness linked to dry skin syndrome or to combat skin irritation following shaving, for example. Any part of the plant and any extraction solvent may be used.

Thus, the above prior art does not exactly suggest that bengle extracts which can be obtained by extraction from rhizomes using at least one apolar organic solvent such as hexane and thus comprising arylbutenoids and/or cassumunarins may have elastin synthesis activation properties, rendering them particularly suitable to topical application to the skin to prevent and/or treat slackening of the skin.

Thus, the present invention provides a cosmetic skin care method to prevent and/or treat at least one sign of skin ageing, comprising topical application to the skin of a composition comprising at least one active ingredient selected from cassumunarin, an arylbutenoid and a botanical extract containing them.

The present invention also encompasses the cosmetic use of a cassumunarin and/or an arylbutenoid and/or a botanical extract containing them to prevent and/or treat at least one sign of skin ageing.

The term “botanical extracts” as used in the present invention means products obtained by extraction using any type of solvent (polar or apolar, liquid or gaseous) from any part of a plant or from the whole plant, in particular from its leaves, flowers, rhizomes, or stems.

Cassumunarins constitute a class of curcuminoids characterized by a structure including a 1-[2-(3-methoxy-4-hydroxy)phenyl 6-(3,4-dimethoxyphenyl]cyclohex-2-enyl penta-2,4-diene-1-one skeleton which differentiate them from curcumin. A, B, and C cassumunarins can be distinguished.

Examples of botanical extracts containing cassumunarins are extracts from bengle (Zingiber cassumunar).

The term “arylbutenoids” as used in the present invention means (non polar) compounds having formula (I) below:

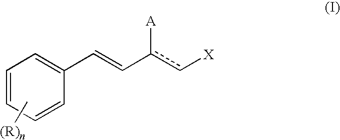

in which:
n represents a whole number from 1 to 3;
R represents a hydroxyl or methoxy group, groups R possibly being identical or different when n is 2 or 3;
A represents a hydrogen atom or a hydroxyl group and X represents a hydrogen atom or a hydroxyl or acetoxy group, or A and X each designate a carbon atom and together form a six-membered ring which may be unsaturated, optionally comprising one or more (R′O)ₘAr substituents where Ar is a phenyl group, R′ is a hydrogen atom or an acetyl group and m is a whole number from 1 to 3; and
the dotted lines represent a bond which may optionally be present.

In formula (I) above, n is preferably 2. Further, groups R are preferably located in the meta- and para-positions on the ring. Preferably again, A and X each represent a hydrogen atom. Finally, when A and X together form a cycle, it is preferably substituted with a dimethoxyphenyl group.

The arylbutenoids of the present invention also include isomers of compounds with formula (I).

Examples of arylbutenoids which may be mentioned in particular are those selected from: 4-(4-hydroxy-3-methoxyphenyl)-3-buten-2-one and extracts from Menta (Litsea sebifera) containing them, as well as (E)-1-(3,4-dimethoxyphenyl)but-1-ene, (E)-4-(3,4-dimethoxyphenyl)buta-1,3-diene, (E)-4-(3,4-dimethoxyphenyl)but-3-en-1-yl acetate, (E)-4-(3,4-dimethoxyphenyl)but-3-en-1-ol, (E)-3-hydroxy-1-(3,4-dimethoxyphenyl)but-1-ene, (E)-4-(4-hydroxy-3-methoxyphenyl)but-3-en-1-yl acetate, 4-(2,4,5-trimethoxyphenyl)but-1,3-diene and extracts from Zingiber cassumunar containing them.

Preferably, in the present invention an extract from bengle or Zingiber cassumunar Roxb or Zingiber purpureum Roscoe is used, which includes both cassumunarins and arylbutenoids.

The botanical extracts which are optionally used in accordance with the invention may in particular be prepared by extraction from the plant under consideration using at least one apolar solvent. This expression means a solvent with a polarity index of less than one which may, for example, be selected from: hexane, cyclohexane, heptane, and isooctane. The apolar solvent may optionally be used as a mixture with at least one polar solvent, generally having a polarity index of more than 3.5, preferably an alcoholic solvent such as ethanol or isopropanol.

In case the plant is bengle, the extract thus obtained is novel.

This invention thus also pertains to an extract from Zingiber cassumunar Roxb, which is obtainable by extracting rhizomes thereof with at least one apolar solvent having a polarity index of less than 1, optionally mixed with at least one polar solvent having a polarity index of more than 3.5.

Preferably, the ratio of the apolar solvent to the polar solvent preferably being in the range 50:50 to 95:5 and preferably in the range 70:30 to 90:10, more preferably in the range 75:25 to 85:15.

Extraction may be carried out on any part of the plant, and with bengle, more particularly from rhizomes which may be dried, which may be ground, or which may be reduced into pieces in the usual manner.

Extraction is generally carried out by gently immersing or agitating the ground material in one or more of the above-mentioned solvents at temperatures which, for example, are from ambient temperature to 100° C., for a period of about 30 minutes to 12 hours (h).

Next, the solution is preferably filtered to eliminate insoluble substances from the plant. If appropriate, the solvent is also eliminated if it is a volatile solvent such as ethanol, methanol, hexane, or cyclohexane, for example.

This extraction step is normal in the plant extract field, and the skilled person can adjust the reaction parameters using general knowledge.

The active ingredient (molecule or botanic extract) used in the present invention may represent 0.00001% to 10% by weight, preferably 0.0001% to 1% by weight, and more preferably 0.001% to 0.1% by weight relative to the total composition weight.

The signs of skin ageing envisaged in the present invention may be signs of chronological ageing (intrinsic) or actinic ageing (photo-ageing). More particularly, the invention aims to combat the cutaneous signs linked to slowing down of the synthesis or degradation of elastin, in particular to prevent and/or treat wrinkles and/or the loss of firmness and/or elasticity of the skin.

Preferably, the active ingredient used in accordance with the invention or the composition used in the method of the invention is applied to the human skin, in particular to the body and/or the face, especially to the area surrounding the eyes. However, it is preferably applied to the body with the exception of the face, in particular to the knees, thighs, hips, buttocks, stomach, and/or arms. It is advantageously applied to subjects showing signs of slackening of the skin.

The composition comprising said active ingredient may be applied morning and/or night, preferably at night, to the zones of the body to be treated, advantageously after rubbing those zones to stimulate microcirculation. Said composition may thus, for example, be applied once a day for four weeks.

Apart from the active ingredient mentioned above, the composition used in accordance with the invention generally includes a physiologically acceptable and in particular a cosmetically acceptable medium, i.e. it does not cause sensations of discomfort (redness, tightness, prickling, etc) which are unacceptable to the consumer.

Said medium generally contains water and optionally other solvents such as ethanol.

The composition used in accordance with the invention may be in any form which is suitable for topical application to the skin, in particular in the form of an oil-in-water, water-in-oil, or multiple emulsion (W/O/W or O/W/W), which may optionally be a microemulsion or a nanoemulsion, or in the form of a hydrodispersion, solution, aqueous gel, or powder. Preferably, said composition is in the form of an oil-in-water emulsion.

Said composition is preferably used as a skin care or cleansing product for the face and/or body and may in particular be in the form of a fluid, gel, or mousse, packaged, for example, in a pump flask, an aerosol, or a tube, or as a cream packaged in a pot, for example. In a variation, it may have the form of a makeup product, in particular a foundation or a loose or pressed powder.

Preferably, the composition of the invention is used as a body care product and is advantageously in the form of a gel or fluid.

It may contain various adjuvants, such as at least one compound selected from:
oils, which may in particular be selected from: linear or cyclic, volatile or non volatile silicone oils such as polydimethylsiloxanes (dimethicones), polyalkylcyclosiloxanes (cyclomethicones), or polyalkylphenylsiloxanes (phenyldimethicones); synthetic oils, such as fluorinated oils, alkylbenzoates and branched hydrocarbons such as polyisobutylene; vegetable oils, in particular soya or jojoba oil; and mineral oils such as paraffin oil;
waxes such as ozokerite, polyethylene wax, beeswax, or carnauba wax;
silicone elastomers, in particular those obtained by reacting, in the presence of a catalyst, a polysiloxane containing at least one reactive group (especially hydrogen or vinyl) and carrying at least one alkyl (especially methyl) or phenyl group, terminally and/or as a side chain, with an organosilicone such as an organohydrogenpolysiloxane;
surfactants, preferably emulsifying surfactants, whether non-ionic, anionic, cationic, or amphoteric, in particular esters of fatty acids and polyols such as esters of fatty acids and glycerol, esters of fatty acids and sorbitan, esters of fatty acids and polyethylene glycol or esters of fatty acids and sucrose; ethers of fatty alcohols and polyethylene glycol; alkylpolyglucosides; polyether modified polysiloxanes; betain and its derivatives; polyquaternium compounds; sulfate salts of ethoxylated fatty alcohols; sulfosuccinates; sarcosinates; alkyl- and di-alkylphosphates and salts thereof; and fatty acid soaps;
co-surfactants, such as linear fatty alcohols, in particular cetyl and stearyl alcohols;
thickeners and/or gelling agents, in particular cross-linked or non cross-linked, hydrophilic or amphiphilic homo- and co-polymers, of acryloylmethylpropane sulfonic acid (AMPS) and/or acrylamide and/or acrylic acid and/or acrylic acid salts or esters; xanthan or guar gum; cellulose derivatives; and silicone gums (dimethiconol);
humectants, such as polyols, including glycerin, propylene glycol and sugars, and glycosaminoglycans such as hyaluronic acid and its salts and esters;
organic filters, such as dibenzoylmethane derivatives (including butyl methoxydibenzoylmethane), cinnamic acid derivatives (including ethylhexyl methoxycinnamate), salicylates, para-aminobenzoic acids, β-β′-diphenylacrylates, benzophenones, benzylidene camphor derivatives, phenylbenzimidazoles, triazines, phenylbenzotriazoles, and anthranilic derivatives; inorganic filters based on mineral oxides in the form of pigments or nanopigments, coated or otherwise, in particular based on titanium dioxide or zinc oxide;
colorants;
preservatives;
fillers, in particular soft-focus effect powders which may in particular be selected from polyamides, silica, talc, mica, and fibers (especially polyamide or cellulose)
tightening agents, in particular plant proteins, synthetic latexes (especially acrylic), and colloidal dispersions of inorganic fillers;
sequestering agents, such as EDTA salts;
fragrances;
and mixtures thereof, this list not being limiting in nature.

Examples of said adjuvants are mentioned in particular in the CTFA Dictionary (International Cosmetic Ingredient Dictionary and Handbook, published by The Cosmetic, Toiletry and Fragrance Association, 9^{th} Edition, 2002).

The composition used in accordance with the invention may also comprise active ingredients other than those described above, in particular at least one active ingredient selected from: agents stimulating the production of growth factors; anti-glycation or deglycating agents; agents augmenting the synthesis of collagen or preventing its degradation (anti-collagenase agents, in particular matrix metalloproteinase inhibitors); agents augmenting the synthesis of elastin or preventing its degradation (anti-elastase agents); agents augmenting the synthesis of glycosaminoglycans or proteoglycans or preventing their degradation; agents augmenting the proliferation or differentiation of keratinocytes; agents augmenting the proliferation of fibroblasts; depigmenting agents, anti-pigmenting agents or pro-pigmenting agents; anti oxidant or anti-radical agents or anti-pollution agents; agents augmenting the synthesis of epidermal lipids; agents stimulating lipolysis, inhibiting lipogenesis and/or inhibiting adipocyte differentiation; draining or detoxing agents or agents which improve microcirculation; and mixtures thereof, this list not being limiting.

Preferably, the composition used in accordance with the present invention comprises at least one agent stimulating lipolysis and/or at least one agent stimulating the synthesis of collagen, more particularly a combination of said agents.

Examples of such additional active ingredients are as follows: plant extracts, in particular extracts from Chondrus crispus, Thermus thermophilus, Pisum sativum, Centella asiatica, Sphacelaria scoparia, Scenedesmus, Moringa pterygosperma, ivy (Hedera helix), Castanea sativa, Hibiscus sabdriffa, Polyanthes tuberosa, Argania spinosa, seeds of Hibiscus esculentus, Narcissus tarzetta, licorice or Ruscus esculatus; an essential oil from Citrus aurantium (Neroli); α-hydroxyacids such as glycolic, lactic or citric acid and esters thereof; β-hydroxyacids such as salicylic acid and its derivatives; silicon derivatives such as methylsilanol mannuronate; plant protein hydrolysates (especially of soya or hazelnut); sugars; acylated oligopeptides, especially those sold by SEDERMA under the trade names Maxilip®, Matrixyl® 3000, Biopeptide® CL or Biopeptide® EL or described in European patent application EP-A-1 449 517; yeast extracts, in particular from Saccharomyces cerevisiae; algae extracts, in particular from laminariae and Blidingia minima; caffeine and coffee and tea extracts containing it; vitamins and derivatives thereof, such as retinyl palmitate, ascorbic acid, ascorbyl glucoside, magnesium or sodium ascorbyl phosphate, ascorbyl palmitate, ascorbyl tetraisopalmitate, ascorbyl sorbate, tocopherol, tocopheryl acetate or tocopheryl sorbate; methacryloyloxyethylphosphorylcholine homo- and co-polymers; urea; ceramides and phospholipids; arbutin; kojic acid; ellagic acid; dihydroxyacetone (DHA); and mixtures thereof.

The invention is illustrated below in the following non-limiting examples.

### EXAMPLES

### Example 1

### Elastin Synthesis Stimulation Test

### a) Protocol

The effect of a bengle (Zingiber cassumunar Roxb) extract on the synthesis of elastin by fibroblasts was investigated using Western Blot.

### Preparation of Extract

The test extract was obtained using the following method.

2.13 g [gram] of bengle (Zingiber Purpureum Roscoe) rhizomes was ground using a cutting mill (RETSCH) then loaded into a 20 liter (L) glass reactor provided with a reflux apparatus. 10 L of a hexane/isopropanol mixture (80/20 v/v) was then added to the milled material. The mixture was allowed to macerate in the cold for two hours, then it was heated for five hours under reflux. After filtering and emptying the reactor, the solvent was evaporated with a vacuum rotary evaporator to recover 0.07694 kg [kilogram] of bengle oleoresin (yield: 3.61%). 502 g of this oleoresin was then distilled by passage through a KDL4 type molecular distillation apparatus (UIC GmbH) using the following distillation parameters:

| Distillation parameters | |
|---|---|
| Insertion rate (mL/h) | 400 |
| Evaporator temperature (° C.) | 125 |
| Condenser temperature (° C.) | 65 |
| Product introduction temperature | 70 |
| (° C.) | |
| Agitation (rpm) | 200 |
| Vacuum pressure (mbar) | 9.5 × 10⁻² |

The distillation residue was recovered then washed with 96.2° ethanol and activated charcoal, at a temperature of 50° C.-60° C., to decolorize it. Next, the filtrate obtained underwent a second washing operation under the same conditions. Next, the final filtrate was filtered on a conical filter to eliminate the activated charcoal residues, and the ethanol was vacuum evaporated.

### In Vitro Test

Fibroblasts (Cambrex) were cultivated for multiplication in a culture medium adapted for fibroblast growth (supplemented DMEM, Gibco), then the fibroblast cultures were seeded into 6-well plates before treating with different concentrations of bengle extract.

The supernatants corresponding to an equivalent number of cells and to an equivalent amount of cellular proteins were fractionated by PAGE gel with a gradient of 8% to 18% (Amersham Pharmacia Biotech, Uppsala, Sweden) and transferred onto nitrocellulose membranes (Schleicher and Schill, Dassel, Germany). Immunodetection was carried out using a monoclonal anti-elastin antibody (Elastin Products Co, PR 533), followed by an HRP-conjugated rabbit anti-goat IgG antiserum (Amersham). The reaction products were detected by chemiluminescence using an ECL kit (Amersham), following the manufacturer's instructions, and quantified by image analysis.

Results: When tested at 50 μg/mL [microgram/milliliter](0.005%) and 100 μg/mL (0.01%), the bengle extract augmented elastin synthesis by 238% on average (compared with the untreated reference) ±70% and by 161% on average ±31% respectively.

It is thus possible to apply said extract topically to the skin to combat the signs of skin ageing.

### Example 2

### Cosmetic Compositions

The following compounds could be prepared in a manner which is conventional for the skilled person. The quantities indicated below are expressed as percentages by weight.

O/W Emulsion

| | |
|---|---|
| Bengle extract* | 0.01% |
| Methylsilanol mannuronate | 5.00% |
| Emulsifying agent (steareth-21) | 1.50% |
| Tocopherol acetate | 0.50% |
| Vegetable oils | 5.00% |
| Silicone oils | 6.00% |
| Octyldodecanol | 2.00% |
| Glycerin | 5.00% |
| Gelling agents | 2.80% |
| Denatured alcohol | 5.00% |
| Sequestering agent | 0.05% |
| pH adjusting agent | qs |
| Preservatives | qs |
| Water | qsp 100.00% |

*prepared as described in Example 1.

This emulsion could be applied every evening to the body to firm it.

Aqueous gel

| | |
|---|---|
| Caffeine | 5.00% |
| Bengle extract* | 0.10% |
| Hydrogenated polyisobutene | 5.00% |
| Emulsifying agents | 5.00% |
| Denatured alcohol | 15.00% |
| Fatty acids | 1.50% |
| Sequestering agent | 0.05% |
| Butylene glycol | 3.00% |
| Gelling agents | 5.00% |
| pH adjuster | qs |
| Preservatives | qs |
| Water | qsp 100.00% |

*prepared as described in Example 1

This gel could be applied to the body morning and evening to firm it and reduce cellulite.

1-17. (canceled)

18. A cosmetic skin care method to prevent and/or treat at least one sign of skin ageing, comprising topical application to the skin of a composition comprising at least one active ingredient selected from cassumunarin, an arylbutenoid, and a botanical extract containing them.

19. The method of claim 18, wherein the cassumunarins are selected from cassumunarins A, B, and C.

20. The method of claim 18, wherein the arylbutenoids have the following formula (I): 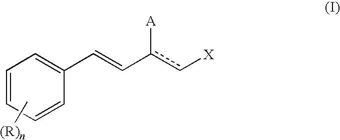
in which:
n represents a whole number from 1 to 3;
R represents a hydroxyl or methoxy group, groups R possibly being identical or different when n is 2 or 3;
A represents a hydrogen atom or a hydroxyl group and X represents a hydrogen atom or a hydroxyl or acetoxy group, or A and X each designate a carbon atom and together form a six-membered ring which may be unsaturated, optionally comprising one or more (R′O)ₘ Ar substituents where Ar is a phenyl group, R′ is a hydrogen atom or an acetyl group and m is a whole number from 1 to 3; and
the dotted lines represent a bond which may optionally be present.

21. The method of claim 20, wherein n equals 2.

22. The method of claim 21, wherein groups R are disposed in the meta- and para-positions on the ring.

23. The method of claim 20, wherein A and X each designate a hydrogen atom.

24. The method of claim 20, wherein A and X together form a cycle substituted with a dimethoxyphenyl group.

25. The method of claim 20, wherein the arylbutenoids are selected from: 4-(4-hydroxy-3-methoxyphenyl)-3-buten-2-one (E)-1-(3,4-dimethoxyphenyl)but-1-ene, (E)-4-(3,4-dimethoxyphenyl)buta-1,3-diene, (E)-4-(3,4-dimethoxyphenyl)but-3-en-1-yl acetate, (E)-4-(3,4-dimethoxyphenyl)but-3-en-1-ol, (E)-3-hydroxy-1-(3,4-dimethoxyphenyl)but-1-ene, (E)-4-(4-hydroxy-3-methoxyphenyl)but-3-en-1-yl acetate and 4-(2,4,5-trimethoxyphenyl)but-1,3-diene.

26. The method of claim 18, wherein the botanical extract is an extract from Zingiber cassumunar Roxb.

27. The method of claim 18, wherein the extract is prepared by extraction using at least one apolar solvent having a polarity index of less than 1, optionally mixed with at least one polar solvent having a polarity index of more than 3.5.

28. The method of claim 27, wherein the apolar solvent is selected from: hexane, cyclohexane, heptane, and isooctane.

29. The method of claim 27, wherein the polar organic solvent is an alcoholic solvent such as ethanol or isopropanol.

30. The method of claim 27, wherein the ratio of the apolar solvent to the polar solvent is in the range 50:50 to 95:5, preferably in the range 70:30 to 90:10, and more preferably in the range 75:25 to 85:15.

31. The method of claim 18, wherein it is intended to combat the cutaneous signs linked to a slowdown in synthesis or degradation of elastin, in particular to prevent and/or treat wrinkles and/or the loss of firmness and/or elasticity of the skin.

32. The method of claim 18, wherein the composition is applied to the human body with the exception of the face, especially to the knees, thighs, hips, buttocks, stomach, and/or arms.

33. An extract from Zingiber cassumunar Roxb, which is obtainable by extracting rhizomes thereof with at least one apolar solvent having a polarity index of less than 1, optionally mixed with at least one polar solvent having a polarity index of more than 3.5.
